(19) United States
(12) Patent Application Publication
(10) Pub. No.: US 2018/0310858 A1
(43) Pub. Date: Nov. 1, 2018

(54) SYSTEM AND METHOD FOR REMOTE DETECTION AND MODULATION OF ELECTRICAL STATES IN LIVING TISSUES USING MAGNETIC PARTICLES AND LIQUID CRYSTALS

(71) Applicant: Weinberg Medical Physics, Inc., North Bethesda, MD (US)

(72) Inventors: Irving N. Weinberg, North Bethesda, MD (US); Lamar Odell MAIR, Baltimore, MD (US); Elisabeth SMELA, Silver Spring, MD (US); Luz MARTINEZ-MIRANDA, North Bethesda, MD (US); Aleksandar Nelson NACEV, Bethesda, MD (US)

(21) Appl. No.: 15/964,289

(22) Filed: Apr. 27, 2018

Related U.S. Application Data
(60) Provisional application No. 62/490,975, filed on Apr. 27, 2017.
(60) Provisional application No. 62/502,208, filed on May 5, 2017.

(51) Int. Cl. A61B 5/055; A61B 5/04; A61K 49/18; G01R 33/381; G01R 33/56

(52) U.S. Cl. CPC A61B 5/055; A61B 5/04005; G01R 33/56; G01R 33/381; A61K 49/1824

(57) ABSTRACT

Disclosed embodiments are directed to a method and system for remotely imaging electric fields from living tissues.

### CROSS-REFERENCE TO RELATED APPLICATION(S)

This application relies for priority on U.S. Provisional Patent Application Ser. No. 62/490,975, entitled “APPARATUS AND METHOD FOR REMOTE DETECTION OF ELECTRIC FIELDS IN LIVING TISSUES USING MAGNETIC PARTICLES AND LIQUID CRYSTALS,” filed on Apr. 27, 2017, and U.S. Provisional Patent Application Ser. No. 62/502,208, entitled “APPARATUS AND METHOD FOR REMOTE DETECTION AND MODULATION OF ELECTRIC FIELDS IN LIVING TISSUES USING MAGNETIC PARTICLES AND LIQUID CRYSTALS,” filed on May 5, 2017, both of which being incorporated herein by reference in their entirety.

### FIELD

Disclosed embodiments are directed, generally, to remote imaging electric fields in living tissue of a subject, human or otherwise. More specifically, disclosed embodiments may be used to remotely characterize the electrical state of living tissue or to modulate the electrical state of living tissue.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Disclosed embodiments are directed to a method and system for remotely imaging electric fields from living tissues.

In accordance with at least one embodiment, a system may include at least one device component containing at least one liquid crystal and at least one magnetic particle, wherein the liquid crystal(s) responds to one or more local electric fields applied to by changing its orientation. Thereby, a change in orientation of the magnetic particle(s) may be produced and controlled.

Further, in accordance with disclosed embodiments, this change in magnetic particle orientation may be detected using Nuclear Magnetic Resonance (NMR), Magnetic Resonance Imaging (MRI), Magnetic Particle Imaging (MPI), or other conventionally known means of remotely detecting changes in magnetic fields.

### BRIEF DESCRIPTION OF FIGURES

A more complete understanding of the disclosed embodiments and the utility thereof may be acquired by referring to the following description in consideration of the accompanying drawings, in which like reference numbers indicate like features, and wherein:

FIG. 1 illustrates an example of a device component of the disclosed system provided in accordance with the disclosed embodiments.

FIG. 2 illustrates an example of a response of the device component to a local electric field in accordance with the disclosed embodiments.

FIG. 3 illustrates an example of a response of the device component to an applied magnetic field in accordance with the disclosed embodiments.

FIG. 4 illustrates an example of method of operations provided in accordance with the disclosed embodiments.

### DETAILED DESCRIPTION

The description of specific embodiments is not intended to be limiting. To the contrary, those skilled in the art should appreciate that there are numerous variations and equivalents that may be employed without departing from the scope of the present invention. Those equivalents and variations are intended to be encompassed by the present invention.

In the following description of various embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

Moreover, it should be understood that various connections are set forth between elements in the following description; however, these connections in general, and, unless otherwise specified, may be either direct or indirect, either permanent or transitory, and either dedicated or shared, and that this specification is not intended to be limiting in this respect.

Early theoretical and experimental work in the field of liquid crystals by de Gennes addressed the orientation of liquid crystal molecules in electric and magnetic fields. See Gennes, P. G. & Prost, J., 1993. The Physics of Liquid Crystals Second Edi., Clarendeon Press, Oxford (incorporated by reference in its entirety). In 1970, Brochard and de Gennes proposed aligning liquid crystal molecules by adding ferromagnetic particles into the liquid crystal solution. See, Brochard, F. & de Gennes, P. G., 1970. Theory of magnetic suspensions in liquid crystals. Journal de Physique, 31(7), pp. 691-708. (incorporated by reference in its entirety).

Garbovskiy demonstrated increases in the switching speed for liquid crystals containing magnetic nanorods, as compared with their pure liquid crystal counterparts. See, Garbovskiy, Y. et al., 2012. Increasing the switching speed of liquid crystal devices with magnetic nanorods. Applied Physics Letters, 101(18)(incorporated by reference in its entirety).

Recent advances in magnetic nanomaterials have yielded magnetically actuated liquid crystals composed of magnetically responsive nanorods. See, Abécassis, B. et al., 2012. Aqueous Suspensions of GdPO4 Nanorods: A Paramagnetic Mineral Liquid Crystal. The Journal of Physical Chemistry B, 116(25), pp. 7590-7595 (incorporated by reference in its entirety). See, also, Wang, M. et al., 2014. Magnetically Actuated Liquid Crystals. Nano Letters, 14, pp. 3966-3971 (incorporated by reference in its entirety).

Magnetic particles have been used extensively as contrast agents for use in medical imaging systems. See, for example, Li, X.-X. et al., 2013. In vivo MRI tracking of iron oxide nanoparticle-labeled human mesenchymal stem cells in limb ischemia. International journal of nanomedicine, 8, pp. 1063-73 (incorporated by reference in its entirety), Ruiz, A. et al., 2013. Short-chain PEG molecules strongly bound to magnetic nanoparticle for MRI long circulating agents. Acta biomaterialia, 9(5), pp. 6421-30 (incorporated by reference in its entirety), Gultepe et al. 2010 (incorporated by reference in its entirety), Bjørnerud, A. & Johansson, L., 2004. The utility of superparamagnetic contrast agents in MRI: theoretical consideration and applications in the cardiovascular system. NMR in biomedicine, 17(7), pp. 465-77. (incorporated by reference in its entirety), and Weissleder, R. et al., 1990 Ultrasmall superparamagnetic iron oxide: characterization of a new class of contrast agents for MR imaging. Radiology, 175(2), pp. 489-493 (incorporated by reference in its entirety).

However, conventionally, the presence of magnetic particles in liquid crystals has not been used to sense electrical states of living tissues using remote instruments sensitive to magnetic properties of materials, or to apply electrical fields to the living tissues. Disclosed embodiments are directed towards this functionality, equipment for providing this functionality and various operations performed to provide the functionality, as disclosed herein.

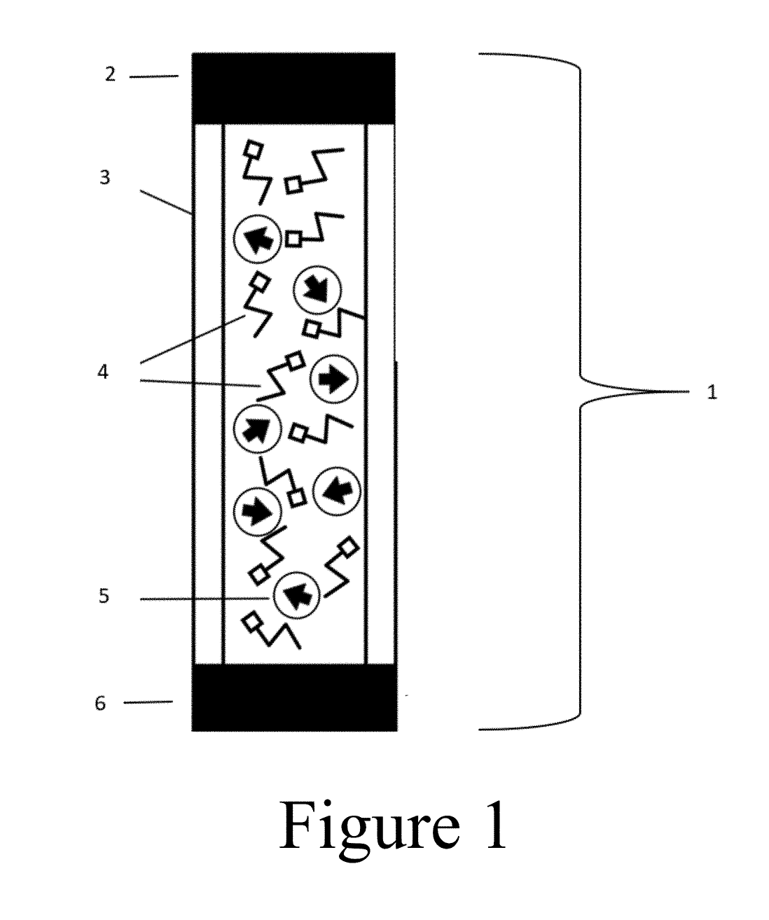

FIG. 1 shows an example of the device component of the apparatus. In this example, the device 1 is in the form of a cylindrical capsule with sidewalls 3 containing both liquid crystal materials 4 and magnetic particles 5 inside the cylindrical capsule. The capsule is shown with electrically conductive caps 2 and 6. FIG. 1 shows the device without an electric field applied to the device.

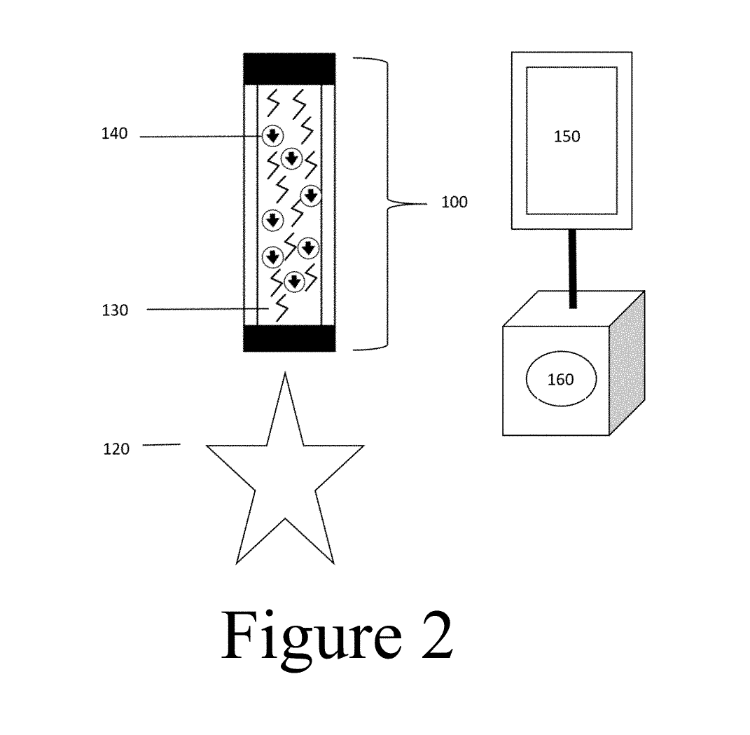

FIG. 2 shows an example of the response of the device 100 to a local electric field from nearby living tissue 120. Electrical signals from nearby components of living tissue 120 cause changes in orientation of the liquid crystal 130, resulting in corresponding changes in orientation of magnetic particles 140. The changes in magnetic particle ordering are detected with at least one sensor 150 that in one embodiment is outside the living tissue. Sensor 150 is connected electrically to computer 160, which can be used to display information about the electrical activity of components of living tissue 120. It is understood that computer 160 includes power supplies, switches, amplifiers, or other electrical or magnetic components as needed to sense the orientations of magnetic particles 140.

The system may include at least one device containing at least one liquid crystal, at least one magnetic particle, the device residing in a living tissue whose properties are to be characterized. The system may include an instrument placed outside said living tissue. For the purposes of this specification, a magnetic particle is defined as a structure smaller than 100 micrometers in any dimension, and containing one or more materials that may be magnetized by an applied magnetic or electromagnetic field. The magnetic particle is smaller than 100 microns in its largest dimension, and may be smaller than 1 micron, and may be smaller than 10 nm, and may be smaller than 1 nm. The device is smaller than 100 microns in its largest dimension, and may be smaller than 1 micron, and may be smaller than 10 nm, and may be smaller than 1 nm. Similarly, individual molecular or particulate components incorporated in the device may have sizes smaller than 1 nm, and may have aspect ratios smaller than 10,000. The device may also contain or be coated with tertiary and quaternary components, being either particles, molecules, or both, which are neither liquid crystal nor magnetic particles. Tertiary and quaternary components may be incorporated into the system as independent components, or may be incorporated into the system via modification or attachment to magnetic particles and/or liquid crystal molecules.

For the purposes of this specification, the liquid crystal component is a material sensitive to electrical fields generated by one or more parts of the living tissue. An example of such electrically-sensitive material is CHDCN. See Korner, H. et al., 1996. Orientation-On-Demand Thin Films: Curing of Liquid Crystalline Networks in ac Electric Fields. Science, 272(5259), pp. 252-255 (incorporated by reference in its entirety). Another example is DOBAMBC (P-(n-(decyloxybenzylidene)-p-amino-2-methylbutyl) Cinnamate), as discussed in Glogarova, M. et al., 1983. The Influence of an External Electric Field on the Structure of Chiral Sm C* Liquid Crystal. Molecular Crystals and Liquid Crystals, 3(4), pp. 309-325 (incorporated by reference in its entirety). See, also Link, D. et al., 1999. Orientation Field Fracture in a Liquid Crystal: Metastable Anticlinic Molecular Tilt in Adjacent Layers in Smectic-C DOBAMBC and TFMHPOBC. Physical Review Letters, 83(18), pp. 3665-3668 (incorporated by reference in its entirety).

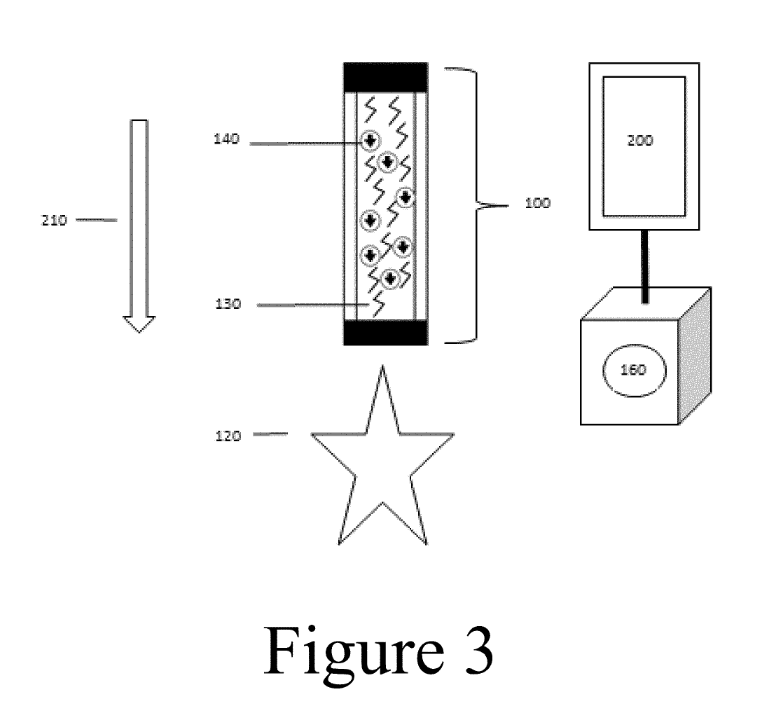

FIG. 3 shows an example of the response of the device 100 to an applied magnetic field 210 produced by at least one coil or magnetic material 200 that in one embodiment is outside the living tissue. For the sake of brevity, we will call part 200 a coil. Coil 200 is connected electrically to computer 160, which can be used to specify the characteristics of magnetic field 210, for example the magnitude, direction, and timing. It is understood that computer 160 includes power supplies, switches, or other electrical or magnetic components as needed to produce magnetic field 210. It is understood that sensor 150 may be part or all of coil 200.

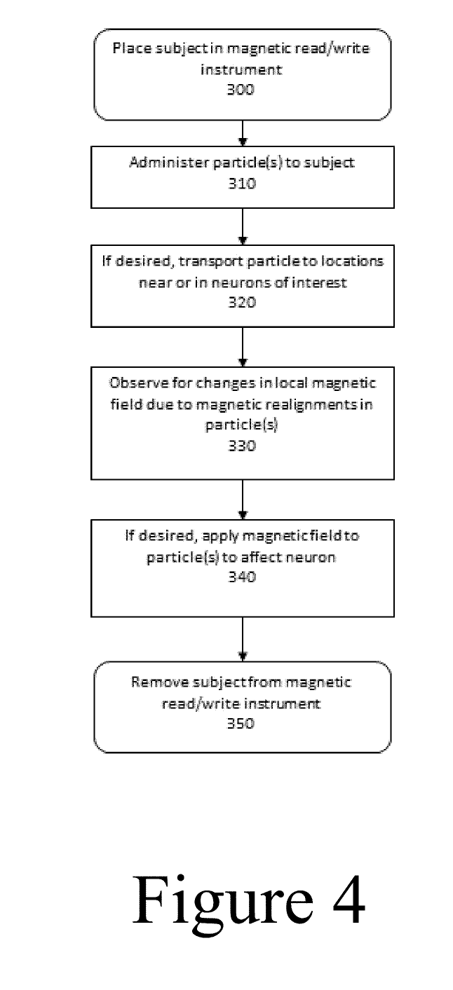

FIG. 4 illustrates an example of the operations performed to provide the functionality disclosed herein. As shown in FIG. 4, in operation 300, a subject is placed in the vicinity of sensor 150 or coil 160 illustrated in other figures. The subject may be a living tissue, a living organ, a human, or other living animal. Subsequently, at 310, one ore more particle device(s) 1 is administered to the subject. This administration may be intra-venous, intra-thecal or may be intra-nasal. In the case of intra-nasal, the administration may be under the influence of a magnetic field by coil 200 or other coils, as taught in the U.S. patent application Ser. No. 13/761,200 by Irving Weinberg entitled “Equipment and Methodologies for Magnetically Assisted Delivery of Therapeutic Agents Through Barriers”, and U.S. patent application 62/553,488 by Irving Weinberg entitled “Noninvasive Treatment for Addiction,” in which magnetic particles are transported via the nose and across the cribriform plate into the brain. Subsequently, at 320, the device(s) may be transported to a location of interest near a neuron or other source of electrical fields by application of magnetic fields to influence movement of a particle, to translate the particle, and/or to rotate the particle. Accordingly, that movement and/or operation may be provided in accordance with the teaching of Aleksandar Nacev, referenced herein and incorporated herein by reference in its entirety.

Subsequently, at 330, the influence of local electric fields generated by tissue 120 cause re-alignments of magnetic particle components 140, which are sensed with the aid of sensor 150 illustrated in other figures. In an option 340, magnetic fields may be applied by coil 200 in order to move or deform device 1. The motion of device 1 may mechanically stimulate tissue 120, as taught in U.S. patent application 62/251,859 by Irving Weinberg, entitled “Apparatus and Methodologies for Neuron Stimulation”, or the deformity of device 1 may cause electrical power production as taught in U.S. patent application Ser. No. 14/221,777 by Irving Weinberg entitled “Apparatus and Method for Spatially Selective Interventional Neuroparticles”, both being incorporated herein in totality. In a subsequent operation 350, the subject is removed from the vicinity of coil 150 or 200.

The present invention describes other means in which neurons or nerves can be affected. As background, it is helpful to note that liquid crystals are present in many living creatures, as taught by D. Chapman in the book “Liquid crystals and cell membranes,” Ann. N. Y. Acad. Sci., vol. 137, no. 2 Biological Me, pp. 745-754, July 1966. Neuronal membranes may be considered to be liquid crystals, and in that role, changes within the liquid crystals lead to altered electrical potentials that can cause neuronal stimulation. It is understood in this invention that the effect on neurons may be stimulatory or inhibitory. Since neurons transmit information via electrical signals, the effect on neurons will be to change the electrical state of the neuron or the tissue that the neuron transmits signal to. It is understood that the term “neuron” is intended to include neurons as well as nerves and other tissues that receive or transmit electrical signals.

It is understood that the alteration of liquid crystal configuration with magnetic particles under the control of a magnetic field applied by an instrument externally to the nervous system of interest may therefore alter the electrical properties of the liquid crystal that is near or in the nervous system, so as to stimulate or otherwise modulate the electrical activity of the nervous system or other components of living tissue. It is understood that the term “electrical properties” in this case includes the permeability to ions, the resistance of the membrane, or the any property which could affect the electrical response of the tissue. An example of the use of magnetic nanoparticles to affect the electric fields from liquid crystals is given by S. Ghosh et al in the scientific paper published in European Letters Association (EPL) on Nov. 10, 2011, volume 96(4), entitled “Effect of multiferroic BiFeO3 nanoparticles on electro-optical and dielectric properties of a partially fluorinated orthoconic antiferroelectric liquid crystal mixture” (Ghosh et al. 2011). Other examples of coupling between the magnetic properties of magnetic particles and the electric properties of liquid crystals are taught in the paper for B. Rozic et al in the publication Molecular Crystals and Liquid Crystals vol. 545(1):pages 99-104, and entitled “Multiferroic Behaviour in Mixtures of the Ferroelectric Liquid Crystal and Magnetic Nanoparticles” (Rožič et al. 2011); and the paper by P. Ganguly et al in Applied Physics Letters 108(18), entitled “Nanoparticles induced multiferroicity in liquid crystal” (Ganguly et al. 2016).

It is understood that such alteration of electrical activity in living tissue could be monitored or otherwise controlled by sensing the voltage in the living tissue using means as described above.

It is understood that the device may be more readily incorporated into components in the living tissue (for example in the neural membrane) through judicious coating of the device (for example with a lipophilic coating as described above).

It is understood that the classes of liquid crystals that may be used to implement the current invention include multiferroic, ferroelectric and antiferroelectric.

In accordance with at least some embodiments, high magnetic gradients may be applied by components 150 or 200 without causing unwanted nerve stimulation, as described in U.S. Pat. Nos. 9,411,030 and 8,466,680 by Irving Weinberg (incorporated herein by reference in their entirety). The magnetic gradients may be used for imaging of the human subject's anatomy and particles and/or components and/or propulsion particles and/or components within the human subject's anatomy, as taught in US Pub. 20130204120 and U.S. Pat. No. 9,380,959 by Irving Weinberg (incorporated herein by reference in their entirety).

In this regard, equipment provided in accordance with the disclosed embodiments may be used to deliver a one or more electric or magnetic fields to a location in a subject's body. Thus, in method implementation, one or more particles may be introduced into tissue within a subject's body and a device for creating one or more forms of Radio Frequency (RF) electromagnetic wave radiation may be provided and placed in proximity to the subject's body or worn on the subject's body. That device 150 and/or 200 for creating RF electromagnetic wave radiation may be any device for generating ambient electromagnetic energy for conversion into electrical energy for use by components, e.g., particle(s) included within a subject's body. For example, the device may be any type of equipment for controlled generating a magnetic field gradient, e.g., MRI machine that applies a magnetic field gradient to subatomic particles in tissue to spatially encode a subsequent response from the atoms and molecules in the tissue to a radiofrequency pulse.

It should be understood that the equipment 150 and 200 for generating magnetic fields may include a magnetic field generator, e.g., a magnetic coil and an RF generator or transmitter. The magnetic coil generates a time-varying magnetic field and the RF generator emits radio waves, and the device may also apply a static magnetic field. The device may include a power source that may be any type of generator suitable for generating power to be provided to the one or more of the components connected thereto.

It should be further understood that the equipment may be implemented within a Magnetic Resonance (MR) device or work in cooperation with one or more MRI devices to provide magnetic fields for positioning and/or manipulation of the particles.

Thus, it should be understood that the device component may operate under control of a controller implemented in whole, or in part, using a computer processor that may be configured assist in performing operations described herein. Accordingly, software code, instructions and algorithms utilized may be utilized by such a processor and may be stored in a memory that may include any type of known memory device including any mechanism for storing computer executable instructions and data used by a processor. Further, the memory may be implemented with any combination of read only memory modules or random access memory modules, optionally including both volatile and nonvolatile memory.

Alternatively, some or all of the device computer executable instructions may be embodied in hardware or firmware (not illustrated). Further, it should be appreciated that, although not illustrated, the controller may similarly be coupled for communication and control to one or more user interfaces that may include display screens, one or more keyboards, and other types of user interface equipment.

Returning to FIG. 2, it should be understood that the at least one device component may be constructed with electro permanent magnets to reduce space and energy consumption, as taught in US patent application U.S. Non-provisional patent application Ser. No. 15/427,426, entitled “METHOD AND APPARATUS FOR MANIPULATING ELECTROPERMANENT MAGNETS FOR MAGNETIC RESONANCE IMAGING AND IMAGE GUIDED THERAPY,” by Irving Weinberg and Aleksandar Nacev (incorporated by reference in its entirety). With such space and energy reductions, it is understood that the apparatus may be readily transported to (and operated in) remote locations.

Finally, in accordance with at least one embodiment, it should be understood that the manipulation of any device component may be performed remotely by a health or medical aid practitioner with access to images of device component and of a subject's body part created by imaging components provided by herein disclosed components.

In accordance with at least one embodiment, the living tissue 120 illustrated in the Figures being investigated and characterized may be an assembly of neurons in an animal or human brain. Likewise, in accordance with at least one embodiment, the electrical activity may be from non-neural sources, for example, a muscle cell.

In accordance with at least one embodiment, one or more devices 100 may be coated with a material (for example, a lipophilic coating) that promotes insertion of at least one portion of the device across a neural membrane so that voltage across the device may be high.

In accordance with at least one embodiment, the one or more devices 100 may be placed less than 100 microns from one or more neurons of the living tissue 120. In accordance with at least one embodiment, electric fields induced by neuronal electrical activity may induce ordering within the liquid crystal component 130 of the at least one device 100. That ordering may be transferred to the magnetic particles 140. See, for example, Martinez-Miranda, L. J. et al., 2006. Effect of the surface coating on the magnetic nanoparticle smectic-A liquid crystal interaction. Applied Physics Letters, 89, p. 161917 (incorporated by reference in its entirety). The degree of magnetic particle ordering in the device 120 may, thereby, be correlated to amplitude of the electric field generated by the neuron.

Transient ordering and disordering of magnetic particles in the device may result in changes in the magnetization of nearby protons. Such changes in magnetization may be detected with NMR or MRI instruments to provide a visual map of the neuronal activity. Alternatively, magnetization of the magnetic particle component of the at least one device 120 may be directly measured using a remote instrument for example using MPI. See, Gleich, B. & Weizenecker, J., 2005. Tomographic imaging using the nonlinear response of magnetic particles. Nature, 435(7046), pp. 1214-7 (incorporated by reference in its entirety).

Such instruments are represented in FIG. 2 as items 150 and 160. It should be understood that one or more components of those instruments (for example, antenna 150) may actually be positioned within the body containing living tissue 120. Alternatively all of the instruments may be external to the body containing living tissues 120.

In an embodiment, the at least one device takes the form of a cylindrical capsule filled with liquid crystal and magnetic particles contained within the capsule. The ends of the cylindrical capsule may contain a conductive material to couple effectively with electric fields in the vicinity of the device. For the purposes of this specification, the term “vicinity of the device” means within a distance of less than 1 mm. This distance may be much smaller, for example one micron or one nanometer. Examples of such conductive materials include gold, platinum, polypyrrole (PPY), polyaniline (PANI), poly(3,4-ethylenedioxythiophene (PEDOT), or a composite of such materials. One or more sections of the device may be composed of an insulating material such as silicon dioxide. One or more sections of the device may be coated with a material that enhances transport across physiological barriers, for example ICAM. See Gultepe, E. et al., 2010. Monitoring of magnetic targeting to tumor vasculature through MRI and biodistribution. Nanomedicine (Lond.), 5(8), pp. 1173-1182 (incorporated by reference in its entirety). See, also, Hsu, J. et al., 2012. Enhanced delivery of α-glucosidase for Pompe disease by ICAM-1-targeted nanocarriers: comparative performance of a strategy for three distinct lysosomal storage disorders. Nanomedicine: nanotechnology, biology, and medicine, 8(5), pp. 731-9. (incorporated by reference in its entirety) for transport across the blood-brain barrier.

One or more sections of the device may be coated with a material to enhance biocompatibility, for example L1 protein. See, for example, Kuo, L. E. et al., 2007. Neuropeptide Y acts directly in the periphery on fat tissue and mediates stress-induced obesity and metabolic syndrome. Nature medicine, 13(7), pp. 803-811. Available at: http://www.ncbi.nlm.nih.gov/pubmed/17603492 (incorporated by reference in its entirety). One or more sections of the device may contain features that enhance propulsion or rotation, as taught by Mair. See, Mair, L. O. et al., 2015. Analysis of driven nanorod transport through a biopolymer matrix. Journal of Magnetism and Magnetic Materials, 380, pp. 295-298 (incorporated by reference in its entirety). See, also, Nacev, A., Stepanov, P. Y. & Weinberg, I. N., 2015. Dynamic Magnetic Inversion Concentrates Ferromagnetic Rods to Central Targets. Nano letters (incorporated by reference in its entirety).

It is understood that in typical operation of the invention, many said devices 100 may be administered to the living tissue. For example, to characterize the electrical activity in the peripheral nerves of a human or other animal, billions of devices may be injected intravenously. The devices might be administered intra-nasally in order to access the brain, as taught by Weinberg. See Weinberg, I. et al., 2012. Non-Invasive Image-Guided Brain Access with Gradient Propulsion of Magnetic Nanoparticles. In IEEE Medical Imaging Meeting. Anaheim, Calif. (incorporated by reference in its entirety).

While disclosed embodiments have been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

It should be understood that, utility of the disclosed system and methodology provide information about electrical activity in a living tissue without a solid connection (e.g., wire). Thus, for the purpose of this disclosure, this property is described as being “untethered.”

Additionally, it should be understood that the functionality described in connection with various described components of various embodiments may be combined or separated from one another in such a way that the architecture of the resulting system is somewhat different than what is expressly disclosed herein. Moreover, it should be understood that, unless otherwise specified, there is no essential requirement that any methodology operations be performed in the illustrated order; therefore, one of ordinary skill in the art would recognize that some operations may be performed in one or more alternative order and/or simultaneously.

Various components of the invention may be provided in alternative combinations operated by, under the control of or on the behalf of various different entities or individuals.

Further, it should be understood that, in accordance with at least one embodiment of the invention, system components may be implemented together or separately and there may be one or more of any or all of the disclosed system components. Further, system components may be either dedicated systems or such functionality may be implemented as virtual systems implemented on general purpose equipment via software implementations.

As a result, it will be apparent for those skilled in the art that the illustrative embodiments described are only examples and that various modifications can be made within the scope of the invention as defined in the appended claims.

We claim:

1. A system comprising:
at least one untethered device component for introduction into living tissue of a subject; and
a controller coupled to equipment for remotely sensing a magnetic field produced by the at least one untethered device component introduced in living tissue of a subject in response to one or more electric fields produced by the living tissue,
wherein the device component is no larger than 100 microns in any single dimension,
wherein the device component contains at least one magnetic particle in a liquid crystal solution,
wherein the liquid crystal is sensitive to one or more electric fields produced by the living tissue, and
wherein the equipment for remotely sensing the configuration of the at least one magnetic particle provides data that enables the system to characterize the electrical status of the living tissue in which the at least one untethered device component is located.

2. The system of claim 1, wherein the untethered device component is positioned within the subject under the influence of magnetic fields produced at least in part by the controller.

3. The system of claim 1, wherein one or more sections of the at least one device component are coated with a biocompatible material.

4. The system of claim 1, wherein one or more sections of the at least one device component are coated with a material to enhance transport to or through living tissue.

5. The system of claim 1, wherein one or more sections of the at least one device component are coated with a material to enhance transport through a barrier between blood and brain.

6. The system of claim 1, wherein the at least one device may be moved or deformed under the influence of magnetic fields produced at least in part by the controller in order to affect the living tissue.

7. The system of claim 1, wherein the at least one device may affect the electrical properties of nearby neurons under the influence of magnetic fields produced at least in part by the controller in order to affect the living tissue.

8. A method comprising:
introducing at least one untethered device component into a living tissue of a subject;
positioning the at least one untethered device component in the living tissue of the subject under control of controller;
applying one or more electric fields produced under control of the controller to the at least one untethered device component in the living tissue of the subject; and
remotely sensing a magnetic field produced by the at least one untethered device component positioned in the living tissue of the subject in response to the one or more electric fields produced by the living tissue,
wherein the device component is no larger than 100 microns in any single dimension,
wherein the device component contains at least one magnetic particle in a liquid crystal solution,
wherein the liquid crystal is sensitive to the one or more electric fields produced by the living tissue, and
wherein equipment for remotely sensing the magnetic field provides data that enables the system to characterize the living tissue in which the at least one untethered device component is positioned.

9. The method of claim 8, wherein one or more sections of the at least one device component are coated with a biocompatible material.

10. The method of claim 8, wherein one or more sections of the at least one device component are coated with a material to enhance transport to or through living tissue.

11. The method of claim 8, wherein one or more sections of the at least one device component are coated with a material to enhance transport through a barrier between blood and brain.

12. The method of claim 8, wherein the at least one device may be moved or deformed under the influence of magnetic fields produced at least in part by the controller in order to affect the living tissue.

13. The method of claim 8, wherein the at least one device may affect the electrical properties of nearby neurons under the influence of magnetic fields produced at least in part by the controller in order to affect the living tissue.

14. A system comprising:
at least one untethered device component for introduction into living tissue of a subject; and
a controller coupled to equipment for remotely sensing a magnetic field produced by the at least one untethered device component introduced in living tissue of a subject in response to one or more electric fields produced by the living tissue,
wherein the device component is no larger than 100 microns in any single dimension,
wherein the device component contains at least one magnetic particle in a liquid crystal solution,
wherein the at least one device affects the electrical properties of at least one nearby neuron under the influence of one or more magnetic fields produced at least in part by the controller.

15. The system of claim 14, wherein the untethered device component is positioned within the subject under the influence of magnetic fields produced at least in part by the controller.

16. The system of claim 14, wherein one or more sections of the at least one device component are coated with a biocompatible material.

17. The system of claim 14, wherein one or more sections of the at least one device component are coated with a material to enhance transport to or through living tissue.

18. The system of claim 14, wherein one or more sections of the at least one device component are coated with a material to enhance transport through a barrier between blood and brain.

19. A method comprising:
introducing at least one untethered device component into a living tissue of a subject;
positioning the at least one untethered device component in the living tissue of the subject under control of controller;
applying one or more electric fields produced under control of the controller to the at least one untethered device component in the living tissue of the subject; and
remotely sensing a magnetic field produced by the at least one untethered device component positioned in the living tissue of the subject in response to the one or more electric fields produced by the living tissue,
wherein the device component is no larger than 100 microns in any single dimension,
wherein the device component contains at least one magnetic particle in a liquid crystal solution,
wherein the at least one device affects the electrical properties of at least one nearby neuron under the influence of one or more magnetic fields produced at least in part by the controller.

20. The method of claim 19, wherein one or more sections of the at least one device component are coated with a biocompatible material.

21. The method of claim 19, wherein one or more sections of the at least one device component are coated with a material to enhance transport to or through living tissue.

22. The method of claim 19, wherein one or more sections of the at least one device component are coated with a material to enhance transport through a barrier between blood and brain.
